(19) United States
(12) Patent Application Publication
(10) Pub. No.: US 2021/0032642 A1
(43) Pub. Date: Feb. 4, 2021

(54) INCREASED ALCOHOL PRODUCTION FROM YEAST PRODUCING AN INCREASED AMOUNT OF ACTIVE HAC1 PROTEIN

(71) Applicant: DANISCO US INC., Palo Alto, CA (US)

(72) Inventors: Joseph Frederich Tuminello, Wilmington, DE (US); Quinn Qun Zhu, West Chester, PA (US); Paula Johanna Maria Teunissen, Palo Alto, CA (US)

(21) Appl. No.: 17/042,569

(22) PCT Filed: Apr. 3, 2019
(86) PCT No.: PCT/US19/25522

§ 371 Date: Sep. 28, 2020

Related U.S. Application Data
(60) Provisional application No. 62/653,858, filed on Apr. 6, 2018.

(51) Int. Cl. C12N 15/81; C12P 7/06

(52) U.S. Cl. CPC C12N 15/81; C12P 7/06

(57) ABSTRACT

Described are compositions and methods relate to modified yeast that produces an increased amount of active HAC1 transcriptional activator involved in the unfolded protein response pathway. Such yeast is well suited for use in fuel alcohol production to increase yield.

### TECHNICAL FIELD

The present compositions and methods relate to modified yeast that produce an increased amount of active HAC1 transcriptional activator involved in the unfolded protein response pathway. Such yeast is well suited for use in fuel alcohol production to increase yield.

### BACKGROUND

Many countries make fuel alcohol from fermentable substrates, such as corn starch, sugar cane, cassava, and molasses. According to the Renewable Fuel Association (Washington D.C., United States), 2015 fuel ethanol production was close to 15 billion gallons in the United States, alone.

Butanol is an important industrial chemical and drop-in fuel component with a variety of applications including use as a renewable fuel additive, a feedstock chemical in the plastics industry, and a food-grade extractant in the food and flavor industry. Accordingly, there is a high demand for alcohols such as butanol and isobutanol, as well as for efficient and environmentally-friendly production methods.

In view of the large amount of alcohol produced in the world, even a minor increase in the efficiency of a fermenting organism can result in a tremendous increase in the amount of available alcohol. Accordingly, the need exists for organisms that are more efficient at producing alcohol.

### SUMMARY

Described are compositions and methods relating to modified yeast that produce an increased amount of active HAC1 transcriptional activator compared to otherwise-identical parental yeast. Aspects and embodiments of the compositions and methods are described in the following, independently-numbered, paragraphs.

1. In one aspect, modified yeast cells derived from parental yeast cells are provided, the modified cells comprising a genetic alteration that causes the modified cells to produce an increased amount of active HAC1 polypeptides compared to the parental cells, wherein the modified cells produce during fermentation an increased amount of alcohol compared to the amount of alcohol produced by the parental cells under identical fermentation conditions.

2. In some embodiments of the modified cells of paragraph 1, the genetic alteration comprises the introduction into the parental cells of a nucleic acid capable of directing the expression of an active HAC1 polypeptide to a level above that of the parental cell grown under equivalent conditions.

3. In some embodiments of the modified cells of paragraphs 1-2, the genetic alteration comprises deletion of a naturally-occurring intron preventing the expression of an active HAC1 polypeptide.

4. In some embodiments of the modified cells of any of paragraphs 1-2, the genetic alteration comprises the introduction of an expression cassette for expressing an active HAC1 polypeptide produced from a genetically-spliced HAC1 gene.

5. In some embodiments of the modified cells of any of paragraphs 1-4, the cells further comprise an exogenous gene encoding a carbohydrate processing enzyme.

6. In some embodiments, the modified cells of any of paragraphs 1-5, further comprise an alteration in the glycerol pathways and/or the acetyl-CoA pathway.

7. In some embodiments, the modified cells of any of paragraphs 1-5, further comprise an alternative pathway for making alcohol.

8. In some embodiments of the modified cells of any of paragraphs 1-7, the cells are of a Saccharomyces spp.

9. In some embodiments of the modified cells of any of paragraphs 1-8, the alcohol is ethanol.

10. In another aspect, a method for increasing the production of alcohol from yeast cells grown on a carbohydrate substrate is provided, comprising: introducing into parental yeast cells a genetic alteration that increases the production of active polypeptides compared to the amount produced in the parental cells.

11. In some embodiments of the method of paragraph 10, the genetic alteration comprises the introduction of a nucleic acid capable of directing the expression of an active HAC1 polypeptide to a level above that of the parental cell grown under equivalent conditions.

12. In some embodiments of the method of paragraph 10 or 11, the genetic alteration comprises deletion of a naturally-occurring intron preventing the expression of an active HAC1 polypeptide.

13. In some embodiments of the method of paragraph 10 or 11, the genetic alteration comprises the introduction of an expression cassette for expressing an active HAC1 polypeptide produced from a genetically-spliced HAC1 gene.

14. In some embodiments of the method of paragraph 10, the cells having the introduced genetic alteration are the modified cells are the cells of any of paragraphs 1-9.

These and other aspects and embodiments of the present compositions and methods will be apparent from the description, including the accompanying Drawings.

### BRIEF DESCRIPTION OF THE DRAWINGS

FIG. 1 illustrates a pre-spliced HAC1 mRNA.

FIG. 2 illustrates a spliced HAC1 mRNA that encoded an active HAC1 protein.

FIG. 3 is a nucleic acid sequence alignment of a pre-spliced HAC1 mRNA, which includes an intron, with the present, genetically-spliced HAC1 mRNA.

### DETAILED DESCRIPTION

### I. Overview

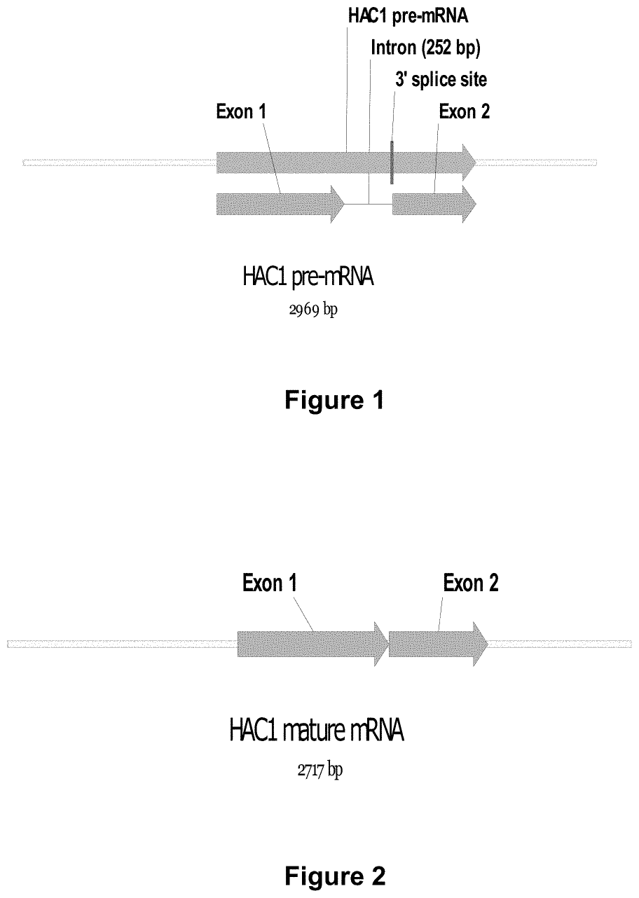

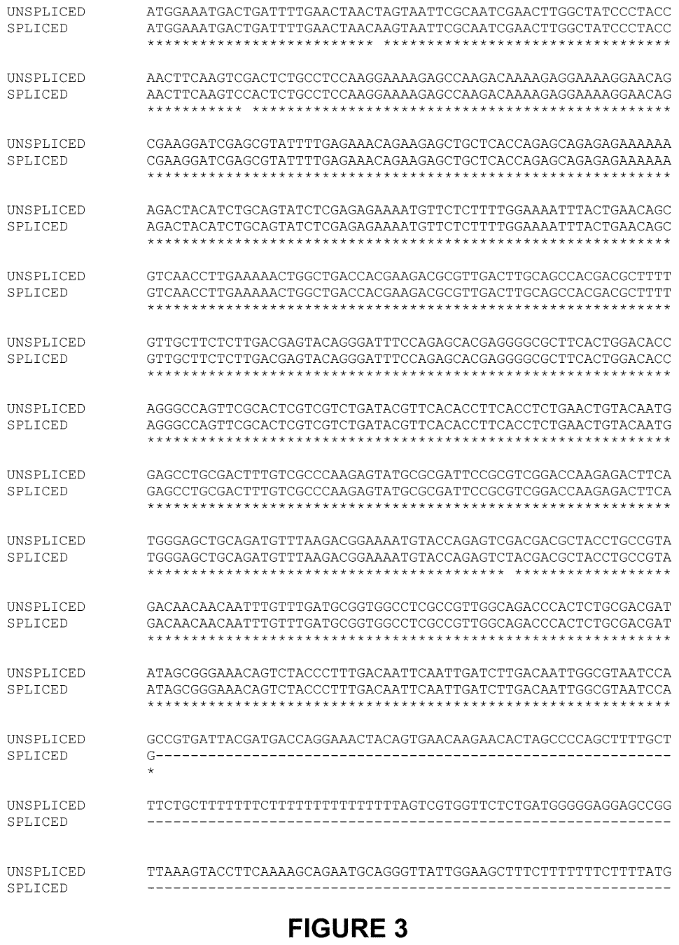

The present compositions and methods relate to modified yeast that produce an increased amount of active HAC1 transcriptional activator involved in the unfolded protein response (UPR) pathway. When translated from a spliced transcript, active HAC1 protein is the mediator of the unfolded protein response, a conserved mammalian and yeast cellular stress response related to endoplasmic reticulum (ER) stress. The native HAC1 gene in Saccharomyces cerevisiae is constitutively transcribed as a message that cannot be translated into active protein due to the presence of an intron (FIG. 1) which, through base-pairing with 5′ UTR, forms a secondary structure that blocks translation initiation. The spliced transcript (FIG. 2) results from ER stress-induced mRNA splicing caused by various environmental stresses.

While the presence of alcohol is known to induce the UPR pathway, it was heretofore unknown that the overexpression of active HAC1 protein, produced from the translation of a “genetically-spliced” mRNA, would result in increased alcohol tolerance. This observation suggests that the UPR pathway in yeast is not naturally optimized for the stress of alcohol production, even in commercial yeast selected specifically for this purpose. Various aspects and embodiments of present composition and methods are described in detail, herein.

### II. Definitions

Prior to describing the modified cells and methods of use in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, “yeast cells,” “yeast strains” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast does not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” or “homolog” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996)Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, “expressing a polypeptide” and similar terms, refer to the cellular process of producing a polypeptide using the translation machinery (e.g., ribosomes) of the cell.

As used herein, “overexpressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes promoter, an amino acid coding sequence, terminator, and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a factor, a co-factor, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, an “active polypeptide/protein” possesses a defined activity.

As used herein, “genetically-spliced,” particularly with respect to a HAC1 gene, transcribed mRNA or active HAC1 protein, refers to a version of the gene, mRNA or protein that has been genetically manipulated by human intervention to exclude any introns responsible for the production of a non-functional HAC1 polypeptide. The term “genetically-spliced” may be substituted with the term “intron-free.”

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
AA α-amylase
bp base pairs
DNA deoxyribonucleic acid
DP degree of polymerization
ds or DS dry solids
EtOH ethanol
g or gm gram
g/L grams per liter
GA glucoamylase
GAU/g ds glucoamylase units per gram dry solids
H₂O water
HPLC high performance liquid chromatography
hr or h hour
kg kilogram
M molar
mg milligram
mL or ml milliliter
ml/min milliliter per minute
mM millimolar
N normal
nm nanometer
PCR polymerase chain reaction
ppm parts per million
RNA ribonucleic acid
Δ relating to a deletion
μg microgram
μL and μl microliter
μM micromolar

### III. Modified Yeast Cells Having Increased Active HAC1 Expression

In one aspect, modified yeast cells are provided, the modified cells having a genetic alteration that results in the production of increased active HAC1 polypeptides (i.e., overexpression of active HAC1) compared to otherwise-identical parental cells. Active HAC1 is an approximately 235-amino acid (typically 230-238) transcription factor found in mammalian cells, yeast and worms. Saccharomyces HAC1 is represented by over twenty publically-available amino acid sequence in GenBank, which are shown in Table 1.

TABLE 1
Saccharomyces HAC1 amino acid sequences in GenBank

| Description | Residues | Accession No. | GI No. |
|---|---|---|---|
| HAC1 [Saccharomyces cerevisiae] | 238 | KZV11568.1 | 1023944339 |
| HAC1 [Saccharomyces cerevisiae] | 230 | BAA05513.1 | 786181 |
| TPA: transcription factor HAC1 | 238 | DAA12409.1 | 285811864 |
| [Saccharomyces cerevisiae S288C] | | | |
| transcription factor HAC1 | 238 | NP_116622.1 | 14318488 |
| [Saccharomyces cerevisiae S288C] | | | |
| RecName: Full = Transcriptional activator HAC1 | 238 | P41546.2 | 115502395 |
| Hac1p [Saccharomyces cerevisiae JAY291] | 230 | EEU04232.1 | 256268883 |
| bZIP protein binding to the CRE motif | 230 | BAA24425.1 | 2804271 |
| [Saccharomyces cerevisiae] | | | |
| conserved protein | 238 | EDN59119.1 | 151940732 |
| [Saccharomyces cerevisiae YJM789] | | | |
| conserved protein | 238 | EDN59119.1 | 151940732 |
| [Saccharomyces cerevisiae YJM789] | | | |
| transcription factor | 230 | GAX72060.1 | 1238273263 |
| [Saccharomyces cerevisiae] | | | |
| transcription factor HAC1 | 230 | PJP09384.1 | 1285342387 |
| [Saccharomyces cerevisiae] | | | |
| Hac1p [Saccharomyces cerevisiae × | 230 | EHN07414.1 | 365765910 |
| Saccharomyces kudriavzevii VIN7] | | | |
| Basic leucine zipper (bZIP) transcription factor | 230 | KQC44186.1 | 941963300 |
| [Saccharomyces sp. ‘boulardii’] | | | |
| HAC1p Basic leucine zipper (bZIP) transcription | 230 | KOH50922.1 | 919303854 |
| factor [Saccharomyces sp. ‘boulardii’] | | | |
| HAC1-like protein | 230 | EJT42879.1 | 401839941 |
| [Saccharomyces kudriavzevii IFO 1802] | | | |
| bZIP protein [Saccharomyces cerevisiae | 238 | EDV09811.1 | 190400394 |
| RM11-1a] | | | |
| HAC1-like protein | 230 | KOG99790.1 | 918733864 |
| [Saccharomyces eubayanus] | | | |
| HAC1-like protein | 230 | KOG99790.1 | 918733864 |
| [Saccharomyces eubayanus] | | | |
| Hac1p [Saccharomyces cerevisiae | 230 | EIW10734.1 | 392299641 |
| CEN.PK113-7D] | | | |
| Hac1p [Saccharomyces cerevisiae | 238 | CAY79417.1 | 259146158 |
| EC1118] | | | |

The amino acid sequence of the exemplified active S. cerevisiae HAC1 polypeptide (i.e., EMBLE Accession No. Z36146.1) is shown, below, as SEQ ID NO: 1:

MEMTDFELTSNSQSNLAIPTNFKSTLPPRKRA
KTKEEKEQRRIERILRNRRAAHQSREKKRLHL
QYLERKCSLLENLLNSVNLEKLADHEDALTCS
HDAFVASLDEYRDFQSTRGASLDTRASSHSSS
DTFTPSPLNCTMEPATLSPKSMRDSASDQETS
WELQMFKTENVPESTTLPAVDNNNLFDAVASP
LADPLCDDIAGNSLPFDNSIDLDNWRNPEAQS
GLNSFELNDFFITS

Based on such BLAST and Clustal W data, it is apparent that the exemplified active S. cerevisiae HAC1 polypeptide shares a high degree of sequence identity to polypeptides from other organisms, and overexpression of functionally and/or structurally similar proteins, homologous proteins and/or substantially similar or identical proteins, is expected to produce similar results to those described, herein.

In particular embodiments of the present compositions and methods, the amino acid sequence of the active HAC1 polypeptide that is overexpressed in modified yeast cells has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

In some embodiments, the increase in the amount of active HAC1 polypeptides produced by the modified cells is an increase of at least 10%, at least 20%, at least 30%, at least 40%, at least 50%, at least 60%, at least 70%, at least 80%, at least 90%, at least 100%, at least 200% or even at least 300% more, compared to the amount of active HAC1 polypeptides produced by parental cells grown under the same conditions.

In some embodiments, the intracellular localization of active HAC1 polypeptides produced by the modified cells is changed, compared to the pattern of active HAC1 polypeptides produced by parental cells grown under the same conditions.

The present composition and methods are not limited to a particular method of overexpression of HAC1; however, because the production of active HAC1 is regulated by splicing, the preferred method for overexpressing HAC1 is to bypass regulation at the splicing level.

The nucleic acid sequence of the exemplified pre-spliced S. cerevisiae HAC1 gene is shown, below, as SEQ ID NO: 2, and illustrated in FIG. 1:

ATGGAAATGACTGATTTTGAACTAACTAGTAAT
TCGCAATCGAACTTGGCTATCCCTACCAACTTC
AAGTCGACTCTGCCTCCAAGGAAAAGAGCCAAG
ACAAAAGAGGAAAAGGAACAGCGAAGGATCGAG
CGTATTTTGAGAAACAGAAGAGCTGCTCACCAG
AGCAGAGAGAAAAAAAGACTACATCTGCAGTAT
CTCGAGAGAAAATGTTCTCTTTTGGAAAATTTA
CTGAACAGCGTCAACCTTGAAAAACTGGCTGAC
CACGAAGACGCGTTGACTTGCAGCCACGACGCT
TTTGTTGCTTCTCTTGACGAGTACAGGGATTTC
CAGAGCACGAGGGGCGCTTCACTGGACACCAGG
GCCAGTTCGCACTCGTCGTCTGATACGTTCACA
CCTTCACCTCTGAACTGTACAATGGAGCCTGCG
ACTTTGTCGCCCAAGAGTATGCGCGATTCCGCG
TCGGACCAAGAGACTTCATGGGAGCTGCAGATG
TTTAAGACGGAAAATGTACCAGAGTCGACGACG
CTACCTGCCGTAGACAACAACAATTTGTTTGAT
GCGGTGGCCTCGCCGTTGGCAGACCCACTCTGC
GACGATATAGCGGGAAACAGTCTACCCTTTGAC
AATTCAATTGATCTTGACAATTGGCGTAATCCA
GCCGTGATTACGATGACCAGGAAACTACAGTGA
ACAAGAACACTAGCCCCAGCTTTTGCTTTCTGC
TTTTTTTCTTTTTTTTTTTTTTTAGTCGTGGTT
CTCTGATGGGGGAGGAGCCGGTTAAAGTACCTT
CAAAAGCAGAATGCAGGGTTATTGGAAGCTTTC
TTTTTTTCTTTTATGCTAGTTTTTCCTGAACAA
ATAGAGCCATTCTTTTCTTATTACTAAGAAATG
GACGGCTTGCTTGTACTGTCCGAAGCGCAGTCA
GGTTTGAATTCATTTGAATTGAATGATTTCTTC
ATCACTTCATGAAGACAATCGCAAGAGGGTATA
ATTTTTTTTTTCTCGTTATTATCGCTGTTGGTG
GGTTTTTTCTTTTCATATATTTCTTTTTCGCTT
AGTGGTTTCTACTGTTCTGTCTCCGGTTAGTGT
GTGCTACTTCAACCGAAGAAGAAGAGGCTTTTC
AAGAATGCAAACGTGAGGTTGGCGCGCCCTCCT
ACAATTATTTGTGGCGACTGGGCAGCGACACTG
AACATAGCTCTTGAACAAGACCCTTTTTTGGCT
GCAAGGAGCAAGACTGGCTGGGGTTCCACCTCA
AAGAGCCACGCTCTGCTTTTTTTCTATCTGTTT
GTGTCATATCTATCTGTCTATTTATCTATATAT
ATATTTTTTTATATAAAACTATAA

The nucleic acid sequence of the exemplified genetically-spliced S. cerevisiae HAC1 gene is shown, below, as SEQ ID NO: 3, and illustrated in FIG. 2:

ATGGAAATGACTGATTTTGAACTAACAAGTAAT
TCGCAATCGAACTTGGCTATCCCTACCAACTTC
AAGTCCACTCTGCCTCCAAGGAAAAGAGCCAAG
ACAAAAGAGGAAAAGGAACAGCGAAGGATCGAG
CGTATTTTGAGAAACAGAAGAGCTGCTCACCAG
AGCAGAGAGAAAAAAAGACTACATCTGCAGTAT
CTCGAGAGAAAATGTTCTCTTTTGGAAAATTTA
CTGAACAGCGTCAACCTTGAAAAACTGGCTGAC
CACGAAGACGCGTTGACTTGCAGCCACGACGCT
TTTGTTGCTTCTCTTGACGAGTACAGGGATTTC
CAGAGCACGAGGGGCGCTTCACTGGACACCAGG
GCCAGTTCGCACTCGTCGTCTGATACGTTCACA
CCTTCACCTCTGAACTGTACAATGGAGCCTGCG
ACTTTGTCGCCCAAGAGTATGCGCGATTCCGCG
TCGGACCAAGAGACTTCATGGGAGCTGCAGATG
TTTAAGACGGAAAATGTACCAGAGTCTACGACG
CTACCTGCCGTAGACAACAACAATTTGTTTGAT
GCGGTGGCCTCGCCGTTGGCAGACCCACTCTGC
GACGATATAGCGGGAAACAGTCTACCCTTTGAC
AATTCAATTGATCTTGACAATTGGCGTAATCCA
GAAGCGCAGTCAGGTTTGAATTCATTTGAATTG
AATGATTTCTTCATCACTTCATGA

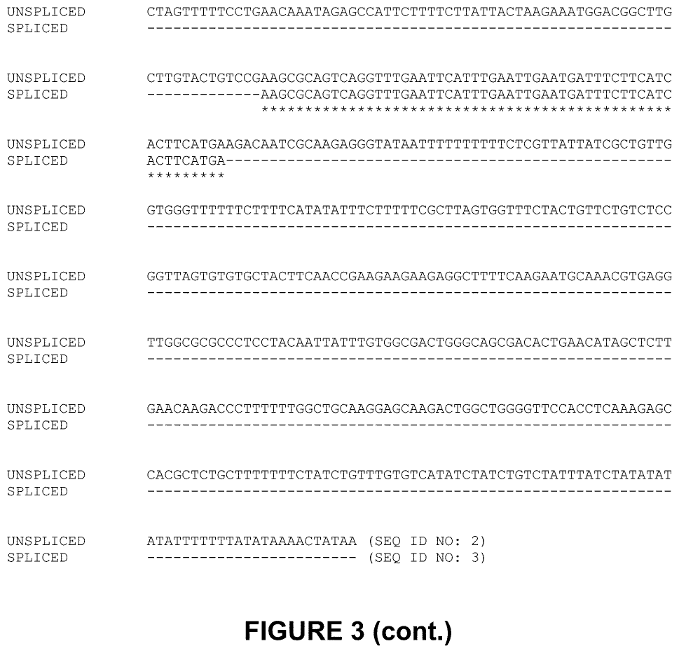

The differences between the pre-spliced and genetically-spliced genes are shown in the Clustal W alignment of FIG. 3. The genetically-spliced gene lacks the intron as well as the native 3′-untranslated region. The genetically-spliced gene also includes an unintentional silent mutation in a threonine codon near the 5′-end.

The deletion of the native 3′-untranslated region is not believed to be relevant to the present compositions and methods. In some embodiments the genetically-spliced gene lacks the intron but includes the 3′-untranslated region. In other embodiments of the present compositions and methods, the genetically-spliced gene lacks the intron as well as the 3′-untranslated region.

In some embodiments, an increased strength promoter is used to control expression of active HAC1 polypeptide produced by the modified cells, optionally in combination with a gene encoding a genetically-spliced active HAC1 protein. The increase in strength may be at least 1-fold, 5-fold, 10-fold, 20-fold, or more, compared to strength of the native promoter controlling HAC1 expression, based on the amount of mRNA produced.

In some embodiments, the increase in alcohol production by the modified cells is an increase of at least 0.3%, at least, 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, at least 2.0% or more, compared to the amount of alcohol produced by parental cells grown under the same conditions.

Preferably, increased active HAC1 production is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the parental cell that is already modified to include a gene of interest, such as a gene encoding a selectable marker, carbohydrate-processing enzyme, or other polypeptide. In some embodiments, a gene of introduced is subsequently introduced into the modified cells.

### IV. Combination of Increased Active HAC1 Production with Genes of an Exogenous PKL Pathway

Increased expression of HAC1 can be combined with expression of genes in the PKL pathway to increase the growth rate of cells and further increase the production of ethanol. Engineered yeast cells having a heterologous PKL pathway have been previously described in WO2015148272 (Miasnikov et al.). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

### V. Combination of Increased Active HAC1 Production with Other Mutations that Affect Alcohol Production and/or Glycerol Reduction

In some embodiments, in addition to expressing increased amounts of active HAC1 polypeptides, the present modified yeast cells include additional modifications that affect ethanol production, or glycerol reduction.

The modified cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al; J Ind Microbiol Biotechnol (2013) 40:1153-1160).

The modified yeast may further feature increased acetyl-CoA synthase (also referred to acetyl-CoA ligase) activity to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further overexpress a sugar transporter-like (STL1) polypeptide to increase the uptake of glycerol (see, e.g., Ferreira et al. (2005) Mol Biol Cell 16:2068-76; Dušková et al. (2015) Mol Microbiol 97:541-59 and WO 2015023989 A1).

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDCS, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### VI. Combination of Increased Active HAC1 Production with Other Beneficial Mutations

In some embodiments, in addition to increased expression of active HAC1 polypeptides, optionally in combination with other genetic modifications that benefit alcohol and/or glycerol production, the present modified yeast cells further include any number of additional genes of interest encoding proteins of interest. Additional genes of interest may be introduced before, during, or after genetic manipulations that result in the increased production of active HAC1 polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transaldolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VII. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VIII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### IX. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1. Materials and Methods

### Liquefact Preparation

Liquefact (ground corn slurry) was prepared by adding 600 ppm of urea, 0.124 SAPU/g ds FERMGEN™ 2.5× (acid fungal protease), 0.33 GAU/g ds variant Trichoderma glucoamylase (TrGA) and 1.46 SSCU/g ds Aspergillus α-amylase, adjusted to a pH of 4.8.

### AnKom Assays

300 μL of concentrated yeast overnight culture was added to each of a number ANKOM bottles filled with 30 g prepared liquefact for a final OD of 0.3. The bottles were then incubated at 32° C. with shaking (150 RPM) for 65 hours.

### HPLC Analysis

Samples from serum vial and AnKom experiments were collected in Eppendorf tubes by centrifugation for 12 minutes at 14,000 RPM. The supernatants were filtered with 0.2 μM PTFE filters and then used for HPLC (Agilent Technologies 1200 series) analysis with the following conditions: Bio-Rad Aminex HPX-87H columns, running temperature of 55° C. 0.6 ml/min isocratic flow 0.01 N H₂SO₄, 2.5 μL injection volume. Calibration standards were used for quantification of the of acetate, ethanol, glycerol, and glucose. Samples from shake flasks experiments were collected in Eppendorf tubes by centrifugation for 15 minutes at 14,000 RPM. The supernatants were diluted by a factor of 11 using 5 mM H₂SO₄ and incubated for 5 min at 95° C. Following cooling, samples were filtered with 0.2 μM Corning FiltrEX CLS3505 filters and then used for HPLC analysis. 10 μl was injected into an Agilent 1200 series HPLC equipped with a refractive index detector. The separation column used was a Phenomenex Rezex-RFQ Fast Acid H+(8%) column. The mobile phase was 5 mM H₂SO₄, and the flow rate was 1.0 mL/min at 85° C. HPLC Calibration Standard Mix from Bion Analytical was used for quantification of the of acetate, ethanol, glycerol, and glucose. Unless otherwise specified, all values are expressed in g/L.

### Example 2. Generation of a Plasmid for the Expression of a Genetically-Spliced HAC1 Gene

The HAC1 coding sequence from S. cerevisiae S288c was synthesized in a “genetically-spliced” form by deleting the intron preventing translation of the native constitutively expressed, inactive HAC1 transcript. The pre-spliced gene, genetically-spliced gene, and a nucleic acid sequence alignment showing the difference between the two genes are illustrated in FIGS. 1-3, respectively. The genetically-spliced gene is represented by SEQ ID NO: 3, shown, below:

ATGGAAATGACTGATTTTGAACTAACAAGTAAT
TCGCAATCGAACTTGGCTATCCCTACCAACTTC
AAGTCCACTCTGCCTCCAAGGAAAAGAGCCAAG
ACAAAAGAGGAAAAGGAACAGCGAAGGATCGAG
CGTATTTTGAGAAACAGAAGAGCTGCTCACCAG
AGCAGAGAGAAAAAAAGACTACATCTGCAGTAT
CTCGAGAGAAAATGTTCTCTTTTGGAAAATTTA
CTGAACAGCGTCAACCTTGAAAAACTGGCTGAC
CACGAAGACGCGTTGACTTGCAGCCACGACGCT
TTTGTTGCTTCTCTTGACGAGTACAGGGATTTC
CAGAGCACGAGGGGCGCTTCACTGGACACCAGG
GCCAGTTCGCACTCGTCGTCTGATACGTTCACA
CCTTCACCTCTGAACTGTACAATGGAGCCTGCG
ACTTTGTCGCCCAAGAGTATGCGCGATTCCGCG
TCGGACCAAGAGACTTCATGGGAGCTGCAGATG
TTTAAGACGGAAAATGTACCAGAGTCTACGACG
CTACCTGCCGTAGACAACAACAATTTGTTTGAT
GCGGTGGCCTCGCCGTTGGCAGACCCACTCTGC
GACGATATAGCGGGAAACAGTCTACCCTTTGAC
AATTCAATTGATCTTGACAATTGGCGTAATCCA
GAAGCGCAGTCAGGTTTGAATTCATTTGAATTG
AATGATTTCTTCATCACTTCATGA

The synthetic HAC1 gene was used to generate plasmid pZX9-HAC1, which contains the HAC1 expression cassette of FBA1 promoter::HAC1::Adh1 terminator. The yeast TDH1 promoter was selected to drive the over-expression of the active form of HAC1. The TDH1 promoter was designed to contain SalI site at its 5′-end and a SpeI site at its 3′-end. The DNA fragment containing the TDH1 promoter was amplified by PCR, and the PCR product was digested with SalI and SpeI. The SalI/SpeI fragment of TDH1 promoter was directionally cloned into plasmid pZX9-HAC1 replacing the FBA1 promoter and completing the spliced HAC1 expression cassette in plasmid pJT805.

### Example 3. Generation of Yeast Overexpressing HAC1

Plasmid pJT805 from Example 2 was used as a template for PCR amplification of the HAC1 expression cassette using appropriate flanking primers having homology to the AAP1 locus of S. cerevisiae. The amplified DNA fragment was used as donor DNA for CRISPR-mediated integration at the AAP1 locus in three parental strains: (i) FG-GA is FERMAX™ Gold Label (Martrex Inc., Minnesota, USA; herein abbreviated, “FG”), a well-known fermentation yeast used in the grain ethanol industry, engineered to expresses an exogenous glucoamylase (GA), (ii) FG-PKL is an engineered FG yeast having a heterologous phosphoketolase (PKL) pathway involving the expression of phosphoketolase (PKL), phosphotransacetylase (PTA) and acetylating acetyl dehydrogenase (AADH), as described in WO2015148272 (Miasnikov et al.), and (iii) FG-PKL-GA is the FG-PKL strain further engineered to expresses an exogenous GA. The exogenous GA in FG-GA and FG-PKL-GA were the same variant of Trichoderma glucoamylase. Integration of the HAC1 expression cassettes were confirmed by PCR.

### Example 4. Effect of HAC1 Over-Expression on Ethanol Production

Two clones of each strain expressing the integrated and overexpressed HAC1 were screened for ethanol production, relative to control strains expressing only native HAC1, by anaerobic growth conducted in Ankom flasks in corn Liquifact growth medium. The ethanol production was analyzed at the end of a 65-hour fermentation. Note that each of the following pairs of data represent independently controlled experiments and should not be compared with each other.

TABLE 2
Ethanol production by variants

| | | Ethanol | Ethanol |
|---|---|---|---|
| Strain | Features | (g/L) | (% increase) |
| FG-GA | GA | 146.48 | -0- |
| G1348 | GA + HAC1 | 147.84 | 0.9 |
| FG-PKL | PKL pathway | 147.07 | -0- |
| GJT23 | PKL pathway + HAC1 | 148.46 | 0.9 |
| FG-PKL-GA | PKL pathway + GA | 147.88 | -0- |
| G1353 | PKL pathway + GA + HAC1 | 148.97 | 0.7 |

Increased ethanol production of 0.7%-0.9% was observed in the HAC1 overexpression strains relative to their parental control strains.

What is claimed is:

1. Modified yeast cells derived from parental yeast cells, the modified cells comprising a genetic alteration that causes the modified cells to produce an increased amount of active HAC1 polypeptides compared to the parental cells, wherein the modified cells produce during fermentation an increased amount of alcohol compared to the amount of alcohol produced by the parental cells under identical fermentation conditions.

2. The modified cells of claim 1, wherein the genetic alteration comprises the introduction into the parental cells of a nucleic acid capable of directing the expression of an active HAC1 polypeptide to a level above that of the parental cell grown under equivalent conditions.

3. The modified cells of claims 1-2, wherein the genetic alteration comprises deletion of a naturally-occurring intron preventing the expression of an active HAC1 polypeptide.

4. The modified cells of any of claims 1-2, wherein the genetic alteration comprises the introduction of an expression cassette for expressing an active HAC1 polypeptide produced from a genetically-spliced HAC1 gene.

5. The modified cells of any of claims 1-4, wherein the cells further comprise an exogenous gene encoding a carbohydrate processing enzyme.

6. The modified cells of any of claims 1-5, further comprising an alteration in the glycerol pathways and/or the acetyl-CoA pathway.

7. The modified cells of any of claims 1-5, further comprising an alternative pathway for making alcohol.

8. The modified cells of any of claims 1-7, wherein the cells are of a Saccharomyces spp.

9. The modified cells of any of claims 1-8, wherein the alcohol is ethanol.

10. A method for increasing the production of alcohol from yeast cells grown on a carbohydrate substrate, comprising: introducing into parental yeast cells a genetic alteration that increases the production of active polypeptides compared to the amount produced in the parental cells.

11. The method of claim 10, wherein the genetic alteration comprises the introduction of a nucleic acid capable of directing the expression of an active HAC1 polypeptide to a level above that of the parental cell grown under equivalent conditions.

12. The method of claim 10 or 11, wherein the genetic alteration comprises deletion of a naturally-occurring intron preventing the expression of an active HAC1 polypeptide.

13. The method of claim 10 or 11, wherein the genetic alteration comprises the introduction of an expression cassette for expressing an active HAC1 polypeptide produced from a genetically-spliced HAC1 gene.

14. The method of claim 10, wherein the cells having the introduced genetic alteration are the modified cells are the cells of any of claims 1-9.


SEQUENCE LISTING (3 sequences)

SEQ ID NO: 1 - protein, 238 aa, Saccharomyces cerevisiae
MEMTDFELTSNSQSNLAIPTNFKSTLPPRKRAKTKEEKEQRRIERILRNRRAAHQSREKKRLHLQYLERKCSLLENLLNSVNLEKLADHEDALTCSHDAFVASLDEYRDFQSTRGASLDTRASSHSSSDTFTPSPLNCTMEPATLSPKSMRDSASDQETSWELQMFKTENVPESTTLPAVDNNNLFDAVASPLADPLCDDIAGNSLPFDNSIDLDNWRNPEAQSGLNSFELNDFFITS

SEQ ID NO: 2 - DNA, 1344 bp, Saccharomyces cerevisiae
atggaaatgactgattttgaactaactagtaattcgcaatcgaacttggctatccctaccaacttcaagtcgactctgcctccaaggaaaagagccaagacaaaagaggaaaaggaacagcgaaggatcgagcgtattttgagaaacagaagagctgctcaccagagcagagagaaaaaaagactacatctgcagtatctcgagagaaaatgttctcttttggaaaatttactgaacagcgtcaaccttgaaaaactggctgaccacgaagacgcgttgacttgcagccacgacgcttttgttgcttctcttgacgagtacagggatttccagagcacgaggggcgcttcactggacaccagggccagttcgcactcgtcgtctgatacgttcacaccttcacctctgaactgtacaatggagcctgcgactttgtcgcccaagagtatgcgcgattccgcgtcggaccaagagacttcatgggagctgcagatgtttaagacggaaaatgtaccagagtcgacgacgctacctgccgtagacaacaacaatttgtttgatgcggtggcctcgccgttggcagacccactctgcgacgatatagcgggaaacagtctaccctttgacaattcaattgatcttgacaattggcgtaatccagccgtgattacgatgaccaggaaactacagtgaacaagaacactagccccagcttttgctttctgctttttttctttttttttttttttagtcgtggttctctgatgggggaggagccggttaaagtaccttcaaaagcagaatgcagggttattggaagctttctttttttcttttatgctagtttttcctgaacaaatagagccattcttttcttattactaagaaatggacggcttgcttgtactgtccgaagcgcagtcaggtttgaattcatttgaattgaatgatttcttcatcacttcatgaagacaatcgcaagagggtataattttttttttctcgttattatcgctgttggtgggttttttcttttcatatatttctttttcgcttagtggtttctactgttctgtctccggttagtgtgtgctacttcaaccgaagaagaagaggcttttcaagaatgcaaacgtgaggttggcgcgccctcctacaattatttgtggcgactgggcagcgacactgaacatagctcttgaacaagacccttttttggctgcaaggagcaagactggctggggttccacctcaaagagccacgctctgctttttttctatctgtttgtgtcatatctatctgtctatttatctatatatatatttttttatataaaactataa

SEQ ID NO: 3 - DNA, 717 bp, Artificial Sequence
  Synthetic
atggaaatgactgattttgaactaacaagtaattcgcaatcgaacttggctatccctaccaacttcaagtccactctgcctccaaggaaaagagccaagacaaaagaggaaaaggaacagcgaaggatcgagcgtattttgagaaacagaagagctgctcaccagagcagagagaaaaaaagactacatctgcagtatctcgagagaaaatgttctcttttggaaaatttactgaacagcgtcaaccttgaaaaactggctgaccacgaagacgcgttgacttgcagccacgacgcttttgttgcttctcttgacgagtacagggatttccagagcacgaggggcgcttcactggacaccagggccagttcgcactcgtcgtctgatacgttcacaccttcacctctgaactgtacaatggagcctgcgactttgtcgcccaagagtatgcgcgattccgcgtcggaccaagagacttcatgggagctgcagatgtttaagacggaaaatgtaccagagtctacgacgctacctgccgtagacaacaacaatttgtttgatgcggtggcctcgccgttggcagacccactctgcgacgatatagcgggaaacagtctaccctttgacaattcaattgatcttgacaattggcgtaatccagaagcgcagtcaggtttgaattcatttgaattgaatgatttcttcatcacttcatga